(19) United States
(12) Patent Application Publication
(10) Pub. No.: US 2024/0325773 A1
(43) Pub. Date: Oct. 3, 2024

(54) MULTIPOLAR MAGNETIC PULSE THERAPY FOR AUTISM AND OTHER NEUROLOGICAL DISORDERS

(71) Applicant: Richard Postrel, Miami Beach, FL (US)

(72) Inventor: Richard Postrel, Miami Beach, FL (US)

(21) Appl. No.: 18/615,871

(22) Filed: Mar. 25, 2024

Related U.S. Application Data
(60) Provisional application No. 63/455,972, filed on Mar. 30, 2023.
(60) Provisional application No. 63/455,971, filed on Mar. 30, 2023.
(60) Provisional application No. 63/463,542, filed on May 2, 2023.

(51) Int. Cl. A61N 2/02; A61N 2/00; G16H 40/60

(52) U.S. Cl. CPC A61N 2/02; A61N 2/006; G16H 40/60

(57) ABSTRACT

This invention discloses a system, method, and device that provides magnetic transcranial stimulation to establish new or adjust existing neuron activities in targeted zones within the brain. This invention corrects neurological pathways that have been damaged, or have dysfunctional neurological systems such as autism, delayed development, and brain trauma, etc. The therapies available using this invention This improved therapy consists primarily of transcranial stimulation (TCS) with precisely targeted magnetic waves rapidly rotating to establish the desired paths of activation. The devices and methods disclosed herein rapidly and painlessly deliver magnetic pulses through the skull to electrically stimulate nerve cells in one or more targeted regions in the brain. The induced electrical activities cause the brain to respond and activate selected cellular pathways in regions of the brain.

### CROSS-REFERENCE TO RELATED APPLICATIONS

This application claims priority to provisional applications 63/455,971 and 63/455,972 filed Mar. 30, 2023 and 63/463,542 filed May 2, 2023, the contents of which are each incorporated herein in their entireties.

### INTRODUCTION

This invention discloses a system, method, and device which allows for variable magnetic flux switching to establish new interneuronal connections or to adjust existing neuronal activities. The device enables precise targeting of selected zones within the brain. An exemplary method employing devices of this invention restores, corrects, replaces, reconfigures, revitalizes, and/or stimulates one or more neurological pathways that have been de-emphasized, damaged, hyperactivated, or present dysfunctional neurological systems such as autism, delayed development, and brain trauma, etc. The therapy disclosed herein leads to expected, normal, reinvigorated, or enhanced brain function(s). The current invention provides significant improvements over current devices and therapies.

This improved therapy consists primarily of transcranial stimulation (TCS) with precisely targeted magnetic waves rapidly rotating to establish the desired paths of activation. The device features one or more sets of paired electromagnet circuits which rapidly and painlessly deliver magnetic pulses through the skull thereby electrically stimulating nerve or supportive cells in one or more targeted regions in the brain. One set or multiple sets of paired electromagnet circuits may be disposed on or around the scalp under direction manually or automatically in accordance with real-time algorithmic messaging or as predetermined based on the subject's criteria. The pulsing magnetic fluxes reorient or drive electrons in the field to electrically stimulate nerve cells in one or more of targeted regions in the brain. The induced electrical activities cause the brain to respond and activate or deactivate selected cellular pathways in regions of the brain that may have become distressed because of, or resulting from, decreased or abnormal activity.

Stimulating glia, non-neuronal cells, can reconfigure neuronal connections and responses and can normalize neuronal responses in the targeted region(s) selected. Different glial cell types characteristically maximally respond to different stimuli (frequency, duration, flux strength, etc) than the neuronal cells or other glial cell types as they alter their activities to reconfigure neuronal (synaptic) connections to normalize the neuronal synapse responses in the targeted region(s). Targeted repetitive magnetic stimulation produces electrical activity in the targeted cells to alter or induce intercellular signaling. The electrical pulse activation at synapses stimulates metabolism, such as ion flux and transport which draws healthful attentions, e.g., blood flow, cell divisions, etc., in the affected region. This induced ion movement and other responses to the targeted stimulus provokes corrective reorganization of neuronal connections within the targeted region. The present invention features therapies for mitigating inapt brain activities. The therapies are multi-component including a collection of monitoring brain functions (and optionally additional organ activities as indicators of brain signaling), and non-invasive cerebral (or cerebellar) stimulation.

### BACKGROUND OF THE INVENTION

Current therapies that employ TCS have been shown to be effective over a variety of syndromes, conditions, diseases, etc.; using various application protocols, devices, frequencies, frequency of sessions and number of repetitions, field strengths, etc. See, e.g., Wieczorek et al 1021 [doi.org/10.12740/PP/OnlineFirst/115556]. O'Connell et al, 2018¹, provided the art with a detailed review studying Repetitive Transcranial Magnetic Stimulation (rTMS) for pain reduction (258 pp.). However, the current invention significantly improves patient outcomes through the use of finely targeted rapid switching (on-off) of opposing magnetic poles stimulating the development of desired healthy pathways. 1 [doi: 10.1002/14651858.CD008208.pub5.]

Magnetic stimulation that is focused or targeted to induce electric currents and cause cells to respond comprise the physical influences that drive the beneficial outcomes from practicing this invention. The delivery of various TCS protocols as the field has developed varied therapies which have produced varied results. The early crude experiments have led to improved and improving devices for generating the electric current inducing magnetic fields that have been applied around and to varied areas of the human skull. While the art has benefited from over a century of experiments and varying therapeutic trials none of these have used rapid switching with rotational stimulation.

In a 2018 review article, O'Connell et al concluded:
“There is very low-quality evidence that single doses of high-frequency rTMS of the motor cortex and tDCS² may have been short-term effects on chronic pain and quality of life but multiple sources of bias exist that may have influenced the observed effects. We did not find evidence of low-frequency rTMS, rTMS applied to the dorsolateral prefrontal cortex and CES are effective for reducing pain intensity in chronic pain. The broad conclusions of this review have not changed substantially for this update. There remains a need for substantially larger, rigorously designed studies, particularly of longer courses of stimulation. Future evidence may substantially impact upon the presented results.”2 transcranial Direct Current Stimulation (tDCS)

Although several peer reviewed reports have demonstrated benefits of magnetic stimulation in treating brain anomalies, there is no consensus on effectiveness. One feature of interest is that treatments described in the art lack provisions for a proper magnet return path. Reports of magnetic stimulus for brain therapies encompass varied strategies. While some therapies have featured static magnetic fields wherein a target is moved or manipulated through the field, many recent therapies have used pulsed electromagnetism to induce an electric current within the pulsed field zone that may include varied portions of the brain tissues. The present invention recognizes these varied strategies and improves on these and other therapeutic strategies to provide novel and effective ameliorative therapies to specifically targeted regions of the brain and thereby to enhance a subject's health and wellness.

### SUMMARY OF THE INVENTION

Generally, the present invention features transcranial stimulation device with at least one pair of magnetic flux generators as a circuit for inducing magnetic fluxes within the brain. Such pair may be focused across a desired path in the brain that includes the area of interest. The pair may be movable to concentrate effect where the fluxes before and after movement intersect. The flux generation is supported by a data processor interface that outputs controlling instructions to one ore more flux generation circuits. Control is facilitated by data from an imaging tool that reports location of induced and responsive activity within the brain. Upon receiving instructive input from the interfaced data processor the flux generation circuit(s) are activated to pulsate magnetic stimulation ton one or more targeted areas or zones in the brain. The data processor works with experience based artificial intelligence (AI) that may access and use data from previous patients or subjects with similar diagnoses or issues, data from earlier sessions of the subject presently undergoing treatment, and instantaneous data from the current treatment session. The AI produces instruction or algorithm to control each pulsation session. The initial frequency within a pulse cluster will generally be instructed in accordance with feedback data from subjects with similar diagnoses or issues. The strength (amplitude) of the pulse spike initially will consider the geometry of the subjects head, the location of the target to be stimulated, and experience data from previous subjects. The pulses within a cluster may present consistent amplitudes through the cluster, may start strong and ramp down, may ramp up, may have a pattern of increasing then decreasing amplitude, and may of course be interrupted if the data processor detects error or unintended neurological response. The AI in each session thus relies on data learned from earlier sessions of several individuals and/or data from the present individual from previous or instantaneous experience. The AI incorporates data indicating what is happening and where the activities are emanating from, e.g., from an imaging device. The instructive algorithms controlling flux generation thus are updated as the AI obtains additional learning and present inputs. Algorithms may be constructed for various effects, including, but not restricted to: determining the next pulse cluster instruction, determining optimal location for a flux generating coil, determining a recovery interpulse interval, changing other inputs to a subject, e.g., when a sound or aroma is used to elicit brain activity. Data processing need not be consigned to a single chip. It may be dispersed, e.g., a coil may feature a dedicated processor actively monitoring and modifying its functions; machinery for positioning a coil may be augmented by a dedicated processor that may include its positional feedback; accessory components, such as Virtual Reality features may include accessory processors. Components that position one or more coils can be present with a track or arm that is interfaced with one or more conductive coils to move or retract each independently.

Repetitive Transcranial Magnetic Stimulation (rTMS) devices of the present invention may incorporate static coils, not moved during t therapeutic session. However, a plurality of coils disposed orthogonally to define individual stimulation signal pathways. Switching stimulation for one pair to a second or third can be practically instantaneous, e.g., on the order of microseconds. By rapidly switching between coil pairs the stimulus can be concentrated at location(s) where paths intersect with lesser stimulus applied to paths leading to or from the specific target. Switching may be predetermined or may be continuously updated from data processing and/or imaging devices serving as accessory components to the coils. Instruction signals to the various device components are selected to avoid interference with data collection and/or flux generation. Imaging data inputs are protected by electronic signal screening that may include active noise cancellation similar to processes used in audio devices, here applied to the electronic signals with no sound waves being outputted. Screening/filtering may be physical and/or electronic. During stimulations the response data emanating from adjacent tissue electrical activity is thus independently sensed from data relevant to stimulus strength from a coil pair. The data emanating from brain tissue electrical activity indicative of strength, frequency and/or source of response is thus separately processable from data relating to the flux generation processes. All data may be incorporated into a machine learning process to contribute to the AI algorithm outputs.

A method to improve patient well-being though adjusting their neurocircuitry is accomplished using a device of the present invention. The subjects status is determined by analyzing signals from a subject's brain to identify one or more ones or areas of abnormal function. The area is inputted into a device of the present invention, e.g., using coordinates obtained from an imaging device. These data are combined in a data processor to create a patient specific algorithm or to access a relevant AI algorithm relating to the individual subject's brain and condition(s) identified. These data and algorithms are compiled to instruct the data processor(s) signal flux generation in accord with the algorithm(s). Following stimulation, response data are collected and outputted into the data processor(s) to optimally adjust the algorithm(s) subjects' continuing therapy.

In one example, a magnetic circuit comprising two magnetic flux inducing coils is situated over the head. The location of each has been predetermined using imaging methods, including, but not limited to: quantitative electroencephalogram MRI, CT, DTI, 3d-EEG, PET, etc. The imaging results for the subject are inputted into a processor that applies AI to instruct coil placement. Once the coils are placed current is applied to induce the desired magnetic flux patterns. The patterns are continuously upgraded or modified in accordance with algorithms produced with AI using real-time imaging feedback obtained during or immediately after each stimulus shot or time defined parameter.

Another preferred embodiment of this invention features a magnetic circuitry with at least 6 electromagnetic coils to provide directed magnetic induced stimulation to precisely targeted points-areas-zones of the brain. Conventional therapies utilize multi-second on-off switching, a rather slow process that does not provide efficient 3-D targeting. The present invention features a device that provides effective 3-D targeting by near instantaneous switching between orthogonally arranged magnetic circuits. The preferred circuitry features a highly specialized conductors and flux generators that allow for rapid switching of the stimuli with low heat generation. Cycling between coil pairs can thus be accomplished on a microsecond scale to allow precise 3-D targeting.

During therapy, real-time monitoring of the brain's electrochemical responses to the therapeutic stimulations provides feedback into the artificially intelligence enhanced control module directing repetitive or modified stimulations. The feedback circuitry and resultant data is processed into and by a machine learning protocol using artificial intelligence (AI) that continuously directs switching between each coil pair, while controlling amplitude, pulsing duration, pulse frequency, interpulse interval, etc., based on the monitored (in real-time) electrical (transmembrane potential) changes at the target.

The stimulations are adjusted according to the ability of the cells at the targeted zone e.g., to release and recycle neurotransmitters, maintain ion gradients, etc. The artificial intelligence algorithms are constructed through multiple session on each individual where the AI incorporates each individual's session into a learned base for guiding subsequent sessions. A library or collection of learned experiences across a population of similarly affected patients is used for early treatments for each patient with the patient's individual responses to each treatment session gradually increasing in dominance. As the library grows, “similarly” will be refined to more closely match an individual's demographics and neurologic manifestations. The continuous monitoring provides important inputs to enable the AI algorithms to process, in real-time, feedback data for controlling the subsequent stimuli. Each algorithm is designed to continue machine learning during each session for each individual. This preferred capacity focuses the therapy to each individual's present needs, i.e., taking into account the current state of the individual's neurometabolisms, including, but not limited to: sleepiness, hormonal influences, general health, hydration, fatigue, distractions, responsiveness of the targeted cells, excitation, etc. The in-session feedback and continuous machine learning allows for shorter, more precisely targeted, and when desired, more frequent session scheduling.

A device/session using individual coils, groups of coils, expanding/contracting stimulus zones, banded zones, dynamic zones, coil pairs, etc. receives contemporary, preferably real time, feedback, applies the developed/selected AI protocols, and reconfigures algorithms for the current and/or a subsequent session to optimize desired/targeted effect. Optimization includes, but is not limited to: pulse amplitude, pulsing durations, pulse frequency, rest times, interpulse intervals, time of day (sleep schedule), distracting or focusing media (VR), shorter sessions, more frequent sessions, improved session effect, improved multi-session effect, an altered or different target zone, etc. Alternate or less optimized embodiments are also useful, for example, especially when an off site or delayed clinical review is desired the updated algorithms may be later finalized or quarantined according to plan. Devices and their operation protocols may thus operate in real time, near real time, or delivered at select delayed intervals. The device may be selected and/or programmed to update algorithms after a number of pulse sessions, therapeutic sessions, or at a specified interval. For example, a device may be set to update algorithms on scales as short as msec, but also over longer spans, e.g., sec, min, hour, day, week, month, season, year. AI producing the algorithms may incorporate learning relating to geo- and/or bio-rhythms, including, but not limited to: sleep schedule, last waking, last feeding, individual circadian rhythm, affect—such as MS, ADD, or ADHD, menstrual cycle, time zone travels, daylight, nighttime, time of day, menstrual cycle, pharmaceutical type and schedule, hormonal—e.g. thyroid assay, even tidal, lunar, seasons, sunspot patterns. The AI is preferably enabled to access many databases in addition to the individual brain activity.

Thus, this disclosure teaches improved therapeutic involvement in reforming brain functions to ameliorate possible degradations, injuries, misdirected signalings, etc.

### BACKGROUND DEVELOPMENT

Several early successes of magnetism in medicine were evident from treating, e.g., shrapnel wounds. In World War I prevalent in the Korean War. Electromagnets might be used to confirm shrapnel location as current fluctuated in response to the shrapnel altering the induced magnetic field. Providing powerful at the time 4 kilowatt magnets were used to treat soldiers who had been injured with steel or iron coated projectiles or splinter fragments and shells or shell casings made with iron or ferro magnetic alloys. The high high currents in these magnets produced a force strength sufficient to extract the foreign material(s). Less powerful magnets are useful for extracting metallic materials from eyes and skin. The directed magnetic forces were less damaging that directed incisions (with a scalpel). And for non-ferrous metals, e.g., brasses and coppers, the shrapnel could often be removed with induced eddy currents in the materials.

Guillaume Duchenne is credited with the recognition of electrophysiology in the 1800s. His electropuncture of muscles and connections to the central nervous system recognized the involvement of electrical currents in controlling many cell functions. Nerves release neurotransmitter chemicals into a synapse to induce an electrical current in the receptive membrane and to activate the receptor cell's actions. The common EKG test that many patients have received is a well-known example where ionic movements in the heart produce an electrical signal that controls the beating heart. Assessing electrical activities in nervous and muscular tissues is used in this and many other diagnostics to this day.

Using magnetism to induce electrical currents and resultant electrophysiological responses soon followed Duchenne's discoveries. An early example of a physiological effect due to a time-varying magnetic field was reported by d'Arsonval in 1896, phosphenes were produced when a volunteer's head was placed inside a coil driven at 42 Hz. Thus, techniques, methods and stimulatory devices have been progressing for more than a century.

At present, TMS apparatus are available in the art. For example, MagStim high-speed stimulator (MagStim Company Limited, Wales, UK) fitted with a round coil (about 60 mm radius) has been proven in several trials.

At present, TMS apparatus are available in the art. For example, MagStim high-speed stimulator (MagStim Company Limited, Wales, UK) fitted with a round coil (about 60 mm radius) has been proven in several trials.

As seen in one published example from the web as observed in January 2023, Brainsway is one provider of Repetitive Transcranial Magnetic Stimulation Treatment (rTMST), for example for obsessive compulsive disorder, Parkinson's Disease, Major Depression Disorder, and Anxious Depression. See e.g., www.brainsway.com/knowledge-center/safety-of-deep-repetitive-transcranial-magnetic-stimulation-drtms-against-medical-refractory-symptoms-in-parkinson-syndromes-first-german-real-world-data-with- a-specific-h5-coil/.
“A 2017 review³ found evidence that an unusual balance between excitatory and inhibitory neurotransmitters may occur during depression, and that the imbalance can be reversed upon depression recovery. The imbalance between excitatory and inhibitory neurotransmitters may be responsible for the reduction in brain volume that occurs in depression according to a 2015 review⁴. 3 Citation from Wilson: Lener M S, Niciu M J, Ballard E D, Park M, Park L T, Nugent A C, Zarate C A Jr. Glutamate and Gamma-Aminobutyric Acid Systems in the Pathophysiology of Major Depression and Antidepressant Response to Ketamine. Biol Psychiatry. 2017 May 15; 81(10):886-897. doi: 10.1016/j.biopsych.2016.05.005. Epub 2016 May 12. PMID: 27449797; PMCID: PMC5107161.4 Citation from Wilson: Lener M S, Iosifescu D V. In pursuit of neuroimaging biomarkers to guide treatment selection in major depressive disorder: a review of the literature. Ann N Y Acad Sci. 2015 May; 1344:50-65. doi: 10.1111/nyas.12759. Epub 2015 Apr. 8. PMID: 25854817.
“In recent years, there's been an interest in identifying brain circuits involved in depression. A brain circuit is essentially a network of neurons. Too much or too little activity in a given circuit may play a role in the onset of depression and other mental health symptoms, according to 2021 research.⁵”5 Citation from Wilson: nLv, Q Y, Chen, M M, Li, Y, Yu, Y, Liao, H. Brain circuit dysfunction in specific symptoms of depression. Eur J Neurosci. 2022; 55(9): 2393-2403. https://doi.org/10.1111/ejn.15221

Sarah Wilson, PhD, Molly Burford: How Does Depression Affect the Brain? www.healthline.com/health/depression-physical-effects-on-the-brain (citing several review articles 2015-2021) Feb. 22, 2022 version accessed Feb. 2, 2023.

rTMS is applicable over a number of diagnosed dysfunctions, including, but not limited to: Post-Traumatic Stress Disorder (PTSD), addiction and dependence syndromes, obsessive-compulsive disorders (OCD), cognitive impairment (including Alzheimer's Disease (AD), Mild Cognitive Impairment (MCI), Lewy bodies dementia, cognitive impairment in the course of Parkinson's Disease (PD)), speech disorder(s) or delay(s), acute phase of both depressive and manic episodes in bipolar disorder type I and potentially type II, schizophrenia, depressions, diabetic neuropathy, chronic central neuropathic pain, facial neuropathic pain, limb neuropathic pain, fibromyalgia, major depressive disorder, traumatic brain injury, prolonged coma, etc. The devices and associated methods provide improved therapeutic systems, for these and other dysfunctions, over those previously applied.

ADHD and autism are just two examples of diseases well-known in the art of diseases to which one or more parts grossly or more subtly have been tagged. Though each affliction/disease may correlate to a particular part, it will manifest in different specific groups of cells in different persons. Accordingly devices, systems and methods of this invention include means for specifically targeting the more dysfunctional groups of cells. Additional applications of AI can be applied to further identify regions that might be expected to contribute to or suffer from the physically identified dysfunctions.

Symptoms of post traumatic stress disorder (PTSD) have been attributed to several key brain regions where altered function is observed in PTSD. The amygdala, the ventromedial prefrontal cortex (vmPFC), and the hippocampus appear to have special involvements. The amygdala is an almond-shaped region centrally located in the brain that appears integral to the normal expression of emotions, especially fear, and reactions to aggressive behaviors, stresses, phobic encounters, and anxiety. The vmPFC appears to be involved in activities such as “emotional processing” and “decision making”. The hippocampus is a large region that coordinates with other parts of the brain to process and retain memories.

Askham, on the Spectrum News website, teaches:
“Children and adolescents with autism often have an enlarged hippocampus, the area of the brain responsible for forming and storing memories, several studies suggest, but it is unclear if that difference persists into adolescence and adulthood.
“The size of the amygdala also seems to differ between people with and without autism, although researchers from different labs have turned up conflicting results. Some find that people with autism have smaller amygdalae than people without autism, or that their amygdalae are only smaller if they also have anxiety. Others have found that autistic children have enlarged amygdalae early in development and that the difference levels off over time.
“Autistic people have decreased amounts of brain tissue in parts of the cerebellum, the brain structure at the base of the skull, according to a meta-analysis of 17 imaging studies. Scientists long thought the cerebellum mostly coordinates movements, but they now understand it plays a role in cognition and social interaction as well.
“Compared with their non-autistic peers, autistic children have significantly faster expansion of the surface area of their cortex from 6 to 12 months of age. In the second year of life, brain volume increases much faster in autistic children than in their non-autistic peers.
“People who lack all or part of one white matter tract called the corpus callosum, which connects the brain's two hemispheres, have an increased likelihood of being autistic or having traits of the condition. The corpus callosum contains many of the long-range connections that extend throughout the brain; the fact that disrupting those connections may lead to autism traits supports the connectivity theory of autism.
“Preschoolers with autism show significant differences in the structure of multiple white-matter tracts, according to a 2020 study. Autistic toddlers and adolescents, too, show alterations in white matter throughout the brain.”

The amygdala is an important structure in the brain's limbic system involved in how animals assess and respond to environmental threats. It appears to have a major role in evaluating the importance of sensory information and prompting an appropriate response. The amygdala is notably involved in regulating emotions, such as fear and aggression. Baseline responses to various inputs vary immensely between individuals. A Feb. 2, 2023 article in the Economist reported studies that recorded activity levels in response to various sensory emotional inputs. The amygdala, striatum, and parts of the cortex dealing with vision hearing and movement were assessed using functional magnetic-resonance imaging. Different responses to different politically charged inputs were correlated with political leanings. This provides clear evidence that tools are available to monitor both high and deep portions of the central nervous system.

“Conversations” between nerves are mediated through chemical neurotransmitters that are released by a sender neuron to one or more recipient (receptor cell(s)). The communication interface between the neurons is called a synapse. An electrochemical signal, aka, an action potential, is mediated by ions (charge carrying atoms). The movement of electrical charges carried by these atoms produces an electric current. At the synapse, the electrical stimulus initiates release of the pre-synaptic (sending) cell's neurotransmitter. As multiple synapses are activated, a stronger signal can be observed. The present invention monitors the activity, ionic potentials, blood flow support, local metabolism, NMR changes observed, etc. to target and monitor zones within the brain as therapeutic targets. Within a specific brain activity center, different neurotransmitters are often active. The present invention does not directly assess specific neurotransmitter release, but may distinguish between one or more based on the observable activations at the activity center. After synaptic activation, metabolism continues. One well-known function comprises removal/recycling of the neurotransmitter chemical. Serotonin transporter (SERT) is highly conserved across species and is a prime target for SSRI drugs. Several dopamine re-uptake inhibitors are pharmacologically utilized to modulate dopamine activity, dopamine being involved in one's ability to stay focused and controlling behaviors especially compulsive activities. Starting or stopping neuro-effective drugs or changes in dose may be useful for mapping target locations for further monitoring and treatments.

Multiple communication systems have developed as life has evolved. The number of neurotransmitters used to communicate between brain cells comprises several score. For example, amino acids (building blocks of proteins) act as simple neurotransmitters between cells, e.g. aspartate, glutamate, glycine, and y-aminobutyric acid (GABA); simple molecules, e.g. NO, CO, CO₂, H₂S, etc., can act as neurotransmitters or hormones; dopamine, epinephrine, norepinephrine, melatonin, serotonin, histamine, tryptamine, phynylethylamine, tyramine, octopamine, 3-iodothyronamine, and N-methylphenethylamine are several well-known compounds in a class of neurotransmitter amines; endogenous cannabinoids such as anandamide exert some activities as neurotransmitters, but also as hormones; ATP and adenosine act as neurotransmitters in some parts of the brain; several peptides including, but not limited to: oxytocin, somatostatin, substance P, and endogenous opioids have both hormonal and neurotransmitter functions. Two of the brain's most common neurotransmitters are glutamate and GABA. These appear to regulate how the brain changes and develops over a lifetime.

For example, when a person experiences chronic stress and anxiety, some of these connections between nerve cells break apart. As a result, communication between the affected cells becomes “noisy,” according to Dr. Krystal. And it's this noise, along with the overall loss of connections, that many believe contribute to the biology of depression.

A person with bipolar disorder experiences manic episodes, or unusually elevated moods in which the individual might feel very happy, irritable, or “up,” with a marked increase in activity level.

### DETAILED DESCRIPTION OF THE INVENTION

Numeric values are approximate, decimal or fractional values are included in approximate expression unless context suggests otherwise. Counts of items are generally exemplary and not intended to exclude values not mentioned. Ranges expressed include the stated limits (minimum and maximum) and all values between “<” and “>” have the mathematical meanings of “less than” and “greater respectively.

The devices and methods of this invention is not restricted to just the conventional applications of rTMS used in brain stimulation. RTMS may be combined with accessory stimuli such as transcranial ultrasound, including Doppler ultrasound. Ultrasound can be applied as a targeted stimulus and may be used to find or pinpoint therapeutic targets, to monitor effects of targeting, to redirect targeting, etc. The ultrasound monitoring and/or stimulation can be used concurrent with rTMS and provide real-time feedback. Transcranial pulsed stimulation featuring ultrasound in a neural stimulation technique can be applied as therapy used to non-invasively target and activate deep brain regions and optionally apply concomitant rTMS/TCS treatment.⁶ 6 “The new treatment is an ongoing scientific development and requires clinicians to have specific neurological and methodological expertise, as well as knowledge of brain function,” explains Beisteiner. Feb. 6, 2021 //neurosciencenews.com/ultrasound-tps-neurology-17695/.

The application of rTMS does not involve electroconvulsive therapy (ECT). Seizures, a primary feature involved in the use of ECT to reset brain miscommunications, are avoided. The devices, systems, and methods described herein result in reworking brain connections and structure for longterm benefit.

An early phase of the invention involves tools and methods that identify targets for improved brain functions. Normal brain function involves communication networks of many interacting cells. Improved brain function involve modifying the neurocircuitry by altering or changing connections in neuronetworks, improving communication between cells, deleting or adding synapses, changing modifying inputs at synapses, etc.

The subject may arrive with a diagnosis that can suggest locations in the brain that historically are associated with the diagnosis, for example a speech defect that historically has been associated with Broca's area (frontal portion of left (dominant) hemisphere, Brodmann areas 44 and 45). Many other cognitive and motor dysfunctions have been associated with specific zones or areas, However, human brains are adaptable to the extreme suggesting that associations between dysfunction and precise anomaly in the brain be confirmed for each subject individual. Though historical teachings can serve as a base for the machine learning and AI algorithms that suggest targets for therapy, the AI parameters are not fixed and precise suggested locations will be overridden when an individual subject provides different data.

A 2021 study of autism spectrum disorder⁷ looking at Brodmann areas 9, 46 and 47 “found that the number of neurons was increased and the number of astrocytes was decreased in layer II of all three prefrontal areas.” The authors noted this was “consistent with a failure of radial glial cells to shift daughter cell production from neurons to astrocytes during prenatal cortical development in autism spectrum disorder.” These recognized anatomical and developmental abnormalities are not targets when simply stimulating or suppressing neuronal activity. Neuronal cells and such support cells can be addressed in accordance with the systems of the present invention to ameliorate a large number of recognized dysfunctions and detectable abnormal activities. 7 Carmen Falcone, et al. Neuronal and glial cell number is altered in a cortical layer-specific manner in autism. Autism: The International Journal of Research and Practice, 25:8 2238-2253, 2021. Abstract. doi.org/10.1177/13623613211014408.

Accordingly, brain scans of each subject are performed to specifically locate therapeutic target(s) for that individual. Scans and results from subsequent subject therapeutic events. A therapeutic event may be an individual treatment session or may comprise multiple sessions on a single day, or repeated sessions over a longer period. The algorithm is updated specific to the individual, preferably prior to each cluster of magnetic pulses, but may be updated in accordance with a session that includes several groups of pulses administered in a session. Algorithms will be improved for each individual as treatment “learning sessions” are compiled with associate results. Algorithms specific to an individual may take account of general learning from a group of individuals, but the individual learning sessions are weighted more strongly than average results.

For each potential treatment plan, the brain is assessed prior to any patient's therapeutic treatments preferably prior to each treatment a subject may receive. Brain “visualization” techniques such as, quantitative electroencephalogram, EEGs. MRIs, PET, Doppler ultrasound, etc., are examples of such imaging protocols known in the art that can provide results to guide targeted application of magnetic pulses in rTMS. Additional stimuli (excitatory or calming), such as sound, odors, images, moving visual images, tactile sensations (e.g., haptic clothing), may be used to more finely target the primary therapeutic zone. After assessing an individual's specific area(s) of dysfunctional activities, applying rTMS to the identified areas(s) may provide stimulus to induce improved activity or may be allied for calming influence. The amplitudes, frequencies, and pulse number are adjustable to concentrate therapy on the identified area, the intended effect, the targeted cells (e.g., astrocyte, neuron) to effect improved function. In preferred embodiments, each stimulus session includes as series of stimulus clusters separated by “rest” intervals with no stimulus or stimulus in another zone or area. The Stimulated portion thus reacts to the cluster both during the stimulus and a recovery period. The recovery period is preferably monitored, e,g., by EEG or cranial activity imaging, allowing automated/algorithmic determination to initiate each cluster.

For example, the pulse frequency is varied according to required effect. Stimulatory rTMS (e.g., between about 5 and 20 Hz), induces increased cortical activity. Where hyperactivity in a portion of the brain requires calming, rTMS is effective when set at a low frequency (generally <1, e.g., 0.25 Hz, but in some individuals in some brain locations up to about 10 Hz). The observed overlap mandates that frequency be adjusted in accord with the individual's responses. Such treatment has been shown to induce a decrease of cortical excitability in most trial subjects. Of course the therapeutic effect on each individual will be observed to confirm intended result. The AI algorithm will adjust one or more, preferably all of frequency, pulsing duration, targeted zone size and shape, rest intervals between pulsing, number of total session, frequency of sessions, etc., to achieve the desired outcomes for each subject individual. Unconsciousness and seizure (according to literature, observed in less than 1 in a thousand subjects) are to be avoided. Parameters that appear to approach or induce either are to be avoided. Induced muscle contraction when a relevant area of the brain is receiving stimulus is managed to avoid physical damage. Durations of pulsations can be varied, e.g, pulses may be applied 1 sec (at 20 Hz—20 pulses), but often for longer periods, e.g., about 2, 3, 5, 7, 10, 12, 15, 20 seconds etc. Rest periods between pulsation sessions can be varied for individual comfort and effect. For example, in some applications the rTMS may induce auditory, visual, olfactory, motor, etc. perceptions. Individual comfort should be taken into account. The interpulse individual allows the synapses at the targeted are to recover, e.g., clear or recycle neurotransmitter, re-energize, re-establish ion gradients, etc. As an example, a series of 3 seconds of pulsations may be separated by recovery periods of about e.g., 10, 15, 20, 30 seconds before a subsequent pulsation session. The rTMS sessions may each continue for several minutes, e.g., about 5, 10, 12, minutes, etc. as tolerated by the subject. For example, an individual expressing symptoms of ADHD may tolerate shorter sessions than an individual exhibiting symptoms of depression. A 12 minute session with pulsation durations of 15 seconds interspersed with 25 second rests would feature 18 pulsation sessions including a terminal rest session that might be include non-magnet energy such as ultrasound used to image changes in structure, circulation, etc. Amplitude is adjusted across the low T range, e.g., single digit, 2, 3, 5, 7, etc., T and, modified in accord with subject response. Peak amplitudes between about 2 and 5 T are readily achievable. Magnetic flux densities can fluctuate rapidly. About 1 T/μsec can be achieved with preferred devices. The fields may reverse polarities, e.g., +yT to −zT as desired. In many instances z=y, but this may be varied. For convenient expression both z and y represent non-numeric values.

One preferred flux configuration has z or y=0; The magnetic field is simply turned off and on. The magnetic pulse from the electromagnet corresponds in shape to the charge change in the electromagnetic coil. For example, the flux density absolute value may ramp up instantaneously, flatten and ramp down instantaneously to form a square shape pulse. The flattened zone may be varied in time as desired. Other shapes are available in accordance in the programming of the electric pulsing. A sinusoidal wave is a common example. Other examples include shapes that may approximate a child's slide—with a steep ramp up and a relaxed down slope. The ramp and slope are not necessarily linear. The derivative of their shapes need not be constant. Thus pulse shapes can be of any shape for example those including, but not limited to common wave shapes such as: square, triangular, sawtooth, sinusoidal, but any variation programmed into the wave generator. The base need not be zero; the field can be biased, e.g., from a weak base field, e.g., 0.2 T with a pulse wave riding over it; perhaps symmetrically from −2.3 T to 2.7 T. The field strengths and variations are at the discretion of the operator or programmer. The pulses may be unidirectional in stimulus, e.g., only north or south magnetic orientation with a simple off mode substituting for the minus or opposite stimulation in for example a sinusoidal or square shape patterning.

The induced currents physically will originate normal to the magnetic field. Since the conductors, nerve fibers, will receive the induced current to cause ion fluxes across the cell membranes and propagation along an axon or other conductive path. The pattern of stimulations can be unidirectional pulsing where opposing coil pairs pulse with the same current directions. Pulses can be all positive—the poles do not reverse; can alternate (with a programmed waveform); can alternate between coils—in the alternation pattern of choice as discussed later; can follow a patterned pulsing, e.g., paradiddle, or other rudiment pattern. The nerve may respond to the alternating simple patterns: 1:1, 2:1, 3:1, etc. ABABABAB, AABAABAAB, AAABAAABAAAB, a rudiment paradiddle: ABAA BABB, or other rudiment—for example—ABAAABAABABBBABB ABAAABAABABBBABB, The pattern is not limited to any simple or more complex rudiment.

The AI is empowered to optimize the algorithmic instruction as learned from previous experiential teachings on similar subjects and/or the current subject and modified by the real-time feedback available during each therapeutic session. The reversing fields have a vibratory effect on the membranes setting up a dynamic potential across membranes that facilitates ion channel opening. The pattern may repeat in an alternating pattern with one or more sets or orthogonal coils. The alternating need not follow a balance with each coil set similarly activated. For example Coil set Z may receive pulsing (including any interpulse intervals) for time Tx; coil set Y may receive pulsing (including any interpulse intervals) for time Ty; coil set W may receive pulsing (including any interpulse intervals) for time Tw; etc.; where Ty and Tz may be equal or not equal; and Tw may or may not equal Ty and/or Tz, etc.

The skin, cutaneous fat, and skull vary immensely between individuals. Accordingly, it is advised that the effect of rTMS be used to calibrate intensity of the pulse. Evoked potentials are often used to monitor effect, especially for motor neurons. Magnetic intensity is adjusted to evoke a potential in a threshold fraction of pulses, for examples 50% to 90% at the discretion of the therapist. A higher % threshold is assumed to affect a greater number of cells in the target zone. During repetitive therapy, the threshold % may be increased or decreased to optimize treatment as therapeutic effects are observed.

As an example, a 20 sec session may include a number of clusters, e.g., 3 clusters 4.5 sec each with a first interpulse interval between clusters 1 and 2 of 2.5 sec, and a second interpulse interval of 4 sec. To further describe the variations the continuous AI contributes to the subject's treatment, an intercluster rest period may extend 10 seconds; the targeting may be recalibrated taking into account data showing a less responsive zone. A second session Seconds, minutes, hours, days, weeks later) may feature a first cluster of 7 sec, an interpulse interval of 3 sec, a second cluster of 3 sec, an interpulse interval of 3 sec, a third cluster of 5.5 sec, an interpulse interval of 7 sec, and a fourth and final session cluster of 11 seconds. The real-time data feedback and continuously updated algorithmic controls of therapy will determine each, the frequency and numbers of each session.

Coincident with the stimulation, the device senses the electro-chemical-biological responses of the brain tissue. The frequencies of the stimulation and of the sensors are selected at different bands to permit filtration/resolution of the electrical responses detected by sensor electrodes to ignore the stimulation frequencies. Such algorithm can include external factors, such as time available, insurance coverage, doctor's orders, etc.

Each session will be of length determined by the individual's algorithm that may include consideration of external factors. One or more pre-session visualizations may be repeated as desired. For example, a PET and or MRI scan may be observed at the outset but only repeated on a periodic schedule or not repeated if therapeutic goals are achieved. The electronic feedback (e.g. EEG like data) provides intersession, intercluster and real-time data controlling each session and contributing to controlling subsequent sessions. Bulk data from a collective of patients can also be used to advise the session AI algorithm.

The AI session algorithms are individualized for each subject and session and cumulatively managed. The session algorithm is the product of several AI iterations. A first level review and analysis is established in accordance with accepted protocols as applied to other patients.

A first level reviews literature, including raw data when available, to construct an atlas of the relevant brain. The first level review will initiate from the several atlases present in the literature for compilation and assimilation. Clinical reports highlight regions of the brain associated with dysfunctions or deficits can direct specific focus to subtle brain zones associated with deficits and compensations. The base atlas includes sub-atlases that use the raw materials to modify the skeleton atlas with characteristics relevant to the intended subject. The first level data include all types including, but not limited to: observation, animal research, organoid, clinical, cell, pharmacological, ablation, etc.

Subject details including, but not limited to: age, skull shape, head size, handedness, previous imaging (MRI, CT, DTI, 3d-EEG, PET, etc.), neurologic symptoms, etc. The AI preferable creates a subject specific sub-atlas as a preliminary working model for each subject.

A second level of AI involvement applies therapeutic data from electrophysiology, TMS, and other interventions. Some of these data were accessed for the first level AI review with weights adjusted accordingly by the AI during its learning phase. The second level AI is instructive in preliminary setup. Coil placement to best access the targeted zone(s), the repetitive intervals for that region and that subject, charge on the coils to induce the magnetic fluxes, the wave shapes and patterns, pulse durations, interpulse patterns, session scheduling, and the like are suggested at this second level. The second level includes subject data from normal volunteers and clinical candidates as well as the level one AI product to formalize an original protocol for the intended subject.

A third level operates individually on each subject and subject session. The preliminary/original protocol is applied. Feedback from the imaging or subject reported result is processed to modulate the stimuli, change the pattern(s), suggest electrode repositioning, etc. Modulation of wave strength, pulse durations, and other stimulus feature are controlled by an AI produced algorithm in rel-time. Algorithms may be multi-component, i.e., comprised of sub-algorithms. A sub-algorithm may determine a signaling pattern to be applied to the subject in a therapy session. It may interface with a second sub algorithm that translates the intended signaling pattern inot machine codes appropriate for relevant components of the magnetic circuits. The machine codes are distributed appropriately to the circuits to be activated at times that may have been arrived at in conjunction with another sub-algorithm. The magnetic circuitry incorporates interface modules or connections to permit manual or AI developed algorithms to instruct activations and functions of components of the intended magnetic circuit in the device. Magnetic circuitry may include a feedback sensor that measures magnetic flux delivered by a circuit and serves to modulate outputs in accordance with intended therapeutic or measuring stimulus.

A fourth level monitors the feedback from the improving stimuli to suggest pausing or finalizing treatment at the first zone, perhaps in favor of a second, third, fourth, etc. zone to treat region(s) that interact with the original target. In some instances the original target will receive subsequent sessions of stimulus.

During therapy, a pulse or series of pulses is administered through a first magnetic circuit. A pulse or series of pulses is administered through a second magnetic circuit. A pulse or series of pulses is administered through a third magnetic circuit. The signals to the three circuits are rapidly interplexed across the three dimensions rapidly switching amongst the pairs with the result that the stimulation from the first circuit is coincident with the other circuit's stimuli on the scale of biologic response. i.e., a cell's depolarization or other membrane response is initiated across the first beam, the second beam intersects with this beam continuing stimulation only at the intersection The third beam reinforces this stimulation. Zones receiving only one circuit's stimulation are only slightly perturbed at a level well below the threshold for response. The system can function with two circuits when desired. However, the three circuit embodiment is preferred because the first and second circuits can therefore be less energized decreasing the risk of stimulation in unintended zones.

In common practice according to the FDA example⁸, the motor threshold level is the minimum stimulator setting, in Standard Motor Threshold pulse or series of pulses is administered through a first magnetic circuit (SMT) units, that induces an observable motor response by the patient in 50% of the applied pulses, usually as observed by movement of the thumb. “MT level” is determined with the rTMS treatment coil positioned over a specific location within the motor strip, called the motor threshold location (MT location). The MT location may be used as an anatomic reference point for navigating the coil to the rTMS treatment location. The MT level is used as a reference point for setting the rTMS treatment intensity, usually expressed as a percent multiple of the MT level, e.g. 120% MT. According to the FDA, rTMS is reasonably safe with mild side effects when performed in compliance with the recommended safety guidelines. The devices, processes, systems and methods of the present invent increase safety and reduce side effects (off target effects) in comparison to conventional technologies. 8 Guidance for Industry and Food and Drug Administration Staff|Class II Special Controls Guidance Document: Repetitive Transcranial Magnetic Stimulation (rTMS) Systems. Document issued on Jul. 26, 2011.

As a base example, in a 2010 J. Clin. Psychiatry paper, Boggio, et al reference a 1998 Grisuru, et al report that used 0.3 Hz stimulation “in lowering the core PTSD symptom of avoidance as well as somatization and symptoms of anxiety and depression.” Boggio also cites additional references as demonstrating effective treatments at 1 and 10 Hz for repetitive TMS is an effective treatment tool. The 2010 Boggio paper reported an apparatus using a figure 8 shaped coil with each wing having a 7 cm diameter.

The present invention expands upon teachings, e.g., of Boggio, by incorporating multiple coils for magnetic induction in a device configured to fit over a subject head. Coils may be of a size much less than the 7 cm of Boggio allowing the device to incorporate multiple coils spread over the dimensions of the head encasing device. Such device may be of any configuration that places the coils in proximity to the cranium. The plurality of coils are used to provide a triangulation effect to effectively and specifically target the regions identified for therapeutic intervention with minimal effect in non-intended areas. Smaller coil sizes can concentrate flux density since the generators are more compact. Coil diameters of about 100 mm produce acceptable concentrated output. Height (thickness of the coil) between about 20 mm and 100 mm, preferably of an amorphous metal for maximized filed strength, produce a strong concentrated flux density.

The coil activities are directed by a power supply providing the electric power (pulses). The output of the power supply to the magnetic flux generating circuit components is controlled in accordance with a session specific algorithm generated by AI in real time from base data and instructions from previous “learning” sessions and modified from the real-time sensor feedback in an ongoing session. Accordingly a data processor operates to combine AI or raw or processed data with real-time output from current session feedback sensors to formulate a session and/or a stimulus activity within a session algorithm. This algorithm is inputted into the module controlling flux generation.

A data processor may comprise installed protocols for AI and data for reprocessing or may receive such inputs from an accessory device or channel. Multiple routines or subroutines may be incorporated within or available to the algorithm outputting processor. The processor has in input for obtaining the real-time progress data of response in or from the brain. Optionally additional data, e.g., subject restlessness, breathing, breath or skin temperature, cardiac data, perspiration, sleepiness, etc. can be available to the AI protocol(s) as the processor formulates and outputs activity and/or session algorithms.

The inventive device that sports the coils may also incorporate components for imaging the brain in the same unit in order to precisely map coil target zones. The coil containing headpiece might be tight fitting headgear or, for example, a frame, pod or cone that fits over the head and from which stimulators—which h may be for imaging, sensors that may receive imaging signal and/or therapeutic coils are dispatched. The live imaging apparatus may be available as an accessory inclusion or device. The coil containing headpiece might be tight fitting headgear, or, for example, a frame, pod or cone, that fits over the head and from which stimulators—which may be for imaging, sensors that may receive imaging signal and/or therapeutic coils are dispatched to approximate the skull.

The term “image” does not require a visual representation. In this context it is simply meant to be a recording of the brain activities or structures mapped in a format that permits accurate reading and targeting. The image can be purely mathematical. The image may deviate from strict 3-D representation, e.g., mapping the brain portions by strength and direction (source) of signal to target the therapeutic zone(s). The brain is imaged, e.g., using electronic, sonic, electromagnetic waves (visual on non-visual spectrum), atomic resonance, etc. to reveal structure and activity zones. Several imaging tools may be coordinated to produce the therapeutic target map, for example, x-ray and EEG may be used to coordinate a physical structure with activity emanating therefrom.

The imaging may result in areas of the brain that appear hyperactive or hypoactive in comparison to normal. The imaging may be used to monitor therapeutic progress when images are compared over a therapeutic time course. To aid the imaging process, stimulants, such as tones, cries, flashing lights, visual images, music of various formats, smells, tactile stimulation (touch, wind, haptic garment, etc), foods, select time of day (e.g., for sleepiness), etc. can be used for circumstantial dysfunctions (e.g., PTSD). The subject is preferably adequately hydrated (for repeatability) and reduced random variance of results from session to session.

The systems and methods described herein relate to related inventions for improving central nervous activity, brain activities. The brain is a complex structure comprising multiple interacting substructures.

For example, the frontal lobe (frontal cortex) plays a significant role managing planning, organizing, strategizing, paying attention to, remembering details, etc. Our frontal cortex is recently evolved and constitutes a comparatively large fraction of our whole brain. The left and right lobes make up a significant portion of what we know as our “thinking” brain, where logical reasoning skills are located, among other activities such as motor sensation, planning, etc. In the frontal lobe, about 30% of the brain is characterized as the prefrontal cortex. This structure has been determined to be involved in important life balancing functions, including, but not limited to: delaying instant gratification, modulating emotions, problem solving, future plans, attention, choosing focus, adapting to present and expected situations, etc. Behind the prefrontal cortex, the frontal cortex harbors the motor cortex including regions dedicated to fine skills, coordinated movements, written word, adversive, aversive, activities, etc.

ADHD exhibits several problems including, but not limited to: inattentiveness, impulsivity, and hyperactivity, that are associated with activities of the prefrontal cortex. Other areas may also be associated. But simply recognizing the precortical involvement would include probably 80% or more of the prefrontal cortex that may not lead to ADHD. And different persons exhibit different ADHD traits to various degrees. Those whose manifestations favored one or more of impulsiveness, forgetfulness, inattentiveness, hyperactivity, ease of distraction, etc. will involve different degrees of dysfunction is different specific parts of the prefrontal cortex and/or other areas in the brain. What sets this general area apart from most others in terms of ADHD research is that it has been shown to be relatively smaller in those with ADHD compared to people without the disorder. The reticular activating system (RAS) has been indicated as affecting or being affected by ADHD. The limbic system and basal ganglia may also be involved in many ADHD incidents. Major Depression Disorder (MDD) is also often associated with portions of the Prefrontal Cortex as well as the amygdala, thalamus, and hippocampus, portions of the brain whose shrinkage correlates with MDD. Higher glucose metabolic activity in the anterior insula as measured using PET was associated with successful pharmacologic intervention while successful behavior therapy was associated with decreased glucose metabolic activity. Activity measurement using an imaging tool is thus recognized to be an important tool for assessing brain dysfunction.

In general historical development of the understanding of our brains, many parts have been given names associated with their locations and structures. Important cultural activities have been associated with some well-identified structures. Accordingly, prime or preferred therapeutic targets may be selected from the hippocampus, the amygdala, the thalamus, the reticular activating system, the frontal cortex including prefrontal cortices, and cerebellum. The hippocampus has been identified in support of memory, learning, navigation, and spacial perception. The thalamus carries coordinating signals from the cerebral cortex, the brain's outer layer, to the brain stem at the brain's center. The amygdala has been associated with memories and emotions. The prefrontal cortex interacts on an emotional level with the amygdala and is involved in cognitive functioning, and impulse control. The reticular activating system (RAS) interplays with the cognitive functioning and impulse in control of obsessive compulsive behavior. Accordingly, this invention recognizes that multiple therapeutic zones may be targeted in a single session or in series across multiple sessions. ADHD in many subjects shows abnormal movements or “motor activities”. The cerebellum is the section of our brain integrately involved in coordinated motor function. It is located some distance from the prefrontal cortex in an area located roughly behind and below the ears.

Cerebellar involvement in ADHD may be as its role as a regulator or “governor” for better control over motor movements, balance, coordination, etc. Not every ADHD subject will express obvious coordination issues. Accordingly, each prospective patient should receive screening involving multiple brain regions.

The precise anatomical regions vary from person to person and activities often overlap across regions. After all the brain organ comprises between 50 and 100 billion interconnecting neurons and their associations with about the same number of glial cells (microglia, astrocytes, oligodendrocytes, and ependymal cells) that support and modify neuronal activities in multiple ways. Altered activities (including, but not limited to: ion balance, neurotransmitter scavenging and recycling, blood supply, hydration, ATP and Ca⁺⁺ release, synthesis of cerebral spinal fluid, mechanical structure, neuronal repair and removal, signal modulation, axon culling, etc.) of glial cells and abnormalities in their numbers have been correlated with several diagnosed brain dysfunctions.

The present invention does not depend on identifying the neurotransmitters involved in the targeted dysfunction. It is brain activities at identified zones that are targeted and monitored. However, although practicing the invention does not require identity of the one or more neurotransmitters party to the targeted dysfunction, when a specific region is targeted or a subject has been shown to be reactive (positively or negatively) to a neurotransmitter, hormone or compounds known the affect such, pharmaceutical intervention modifying the identified compound may be employed to aid targeting or to augment therapy.

To proceed with remediation therapy in an individual, a targeted function is selected. For example, for post-stroke therapy, the stricken zone and/or its remaining functioning neuro-connected centers may be multiple targets. MRI, PET, 3D-EEG, or other imaging protocols are useful for selecting targets. Stimulating the stricken zone enhances nerve repair processes. Stimulating centers that normally transmit signal to the stricken zone can induce recipient nerves present there (even if efferent pathways are not apparent) to initiate or enhance repair processes. Stimulating centers that receive nerve impulses from the stricken zone can activate loops feeding back to the stricken zone.

### ILLUSTRATIVE EXAMPLE

A targeted function or diagnosis is selected. For example, for post-stroke therapy, the stricken zone and/or its remaining functioning neuro-connected centers may be multiple targets. MRI, PET, 3D-EEG, CT, PET-CT, Doppler, or other imaging protocols are useful for selecting targets with or without contrast. Stimulating the stricken zone enhances nerve repair processes. Stimulating centers that normally transmit signal to the stricken zone can induce recipient nerves present there (even if efferent pathways are not apparent) to initiate or enhance repair processes. Stimulating centers that receive nerve impulses from the stricken zone can activate loops feeding back to the stricken zone.

Eltagy, et al provides one exemplary teaching demonstrating the availability of aspects of TCS treatments in conjunction with imaging to accurately target zones for remediation. Positron Emission Tomography (PET) is a precise method for mapping brain activities. Positional electroencephalography is adequate for directing the zones and intensities of magnetic bursts used for TMST.

Application of soundwaves, ultrasound, has two applications in the present invention. The ultrasound itself can be applied as an augmenting stimulus, i.e., a stimulus applied concurrent with the TMST, in the interburst interval, and/or ion a separate session. Ultrasound is also available for imaging, e.g., to monitor and confirm progress in the applied therapy. Where specific functions are targeted for remediation, such as hand, the relevant homunculus hand would be the selected target. Confirmation of the cortical target can be achieved by monitoring hand stimulation or motor function while recording and identifying the cortical zone responsible for the sensation or activity. Similar practice can be performed over the cerebellum when the subject would benefit. Where centers may be changed by accident and recovery or from a congenital condition, the compensatory location of the center might be confirmed by eliciting action by the subject. For example, where a subject may have compensated for a congenital defect, e.g., with dysphasia or dyslexia, the compensating zones are confirmed by, e.g., asking the subject to read and/or speak.

After identifying the target zone and result, e.g., activation or calming, the rTMS commences.

Delivering the magnetic pulses can be through a coil or coils strategically positioned over the scalp to magnetically target the selected zone(s). Such coils may reside over and/or around the head where the head is stationarily placed in proximity to a frame supporting coils. Coils may be in a flexible cap or other covering that fits over the subject's head. A mask or container surrounding the head may hold the coils for therapy. Multiple coils may be incorporated into a device, with specific coils being selected for a specific subject or specific subject session.

VR programming is selected to augment the desired effect of the TMS. For example, when a calming rTMS is used, calming sounds and scenes are featured in the VR outputs. The panoptic device is preferably configured to provide an aroma therapeutic oil e.g., an oil selected selected from essential oils including, but not limited to: rose, lavender, vetiver, basil, sage, frankincense, geranium, lemon grass, lemon balm, chamomile, ylang ylang, patchouli, marjoram, melissa, rosemary, bergamot, etc. The practitioner is advised to test each subject's response to one or more candidate oils because individual's experiences may have associated specific events, stressful or relaxing with an oil or combination. Aroma therapy may be enhanced by or replaced with one or more other calming or excitatory influences, e.g., music, visual scenes or patterns, masking such as a white noise, etc. As with the oils, such therapeutic contributors should be confirmed for intended effect with each subject.

The format of the device is not fixed. For example, a partial or full body massage chair fitted with a VR display is one embodiment, e.g., a Virtually Reality (VR) mask or egg, structure may be used. The massage may be coincident with the pulse therapies, may be constant during and between pulsed therapy bursts, may turn on or increase between bursts, etc. Massage may involve human interaction, for example with a masseuse whose presence may provide physical and/or emotional stimulation to assist in therapeutic screening and/or effect. Massage may be by human or mechanical means. It may comprise skin on skin; glove on skin, liquid or gas on skin, etc. For example, a pneumatic tube or sleeve may be disposed on a body part, e.g., a limb—arm or leg, an extremity, e.g., foot, hand, finger(s), toe(s). The pneumatic accessory may simply pulse its entire massage zone or may be programmed to pulse along its length. The cap or head covering may incorporate physical simulation as well as provide the magnetic flux stimulation. The “massage” may be intertwined with VR or ER to enhance therapy.

Hyperoxygenation, e.g., using a hyperbaric chamber, a mask delivering enhanced O₂ concentrations in concert with aromatherapeutic delivery, an enclosed VR module outfitted to control O₂ concentration, etc. In place of on in concert with a massaging module, embodiments may include apparel such as gloves, vests, leggings that provide augmenting sensations, e.g., during a VR adventure, as additional stimulus to synergize with the TMS bursts, etc. For example, sensory (haptic) gloves may coordinate with the VR adventure for increased reality, may augment the TMS bursts to coordinate physically stimulation with the magnetic. VR outputs, auditory, visual, olfactory, etc., can be integral with sensing and/or stimulus devices, systems, or components. For example, a helmet can cover the head, provide sound and visual outputs prior to and/or during sensing (imaging), which may be coincident with, interspersed between and/or pre- or post-therapeutic session(s). A preferred sensing device is a device that functions in real-time during or in close time proximity to stimulus. A 3d-EEG system is one well-known such device that is capable of providing feedback during a stimulus session at times between stimulus activities and during activity where frequencies are selected to permit differential filtering. The in session feedback is considered by the AI, especially at the third level to adjust the algorithm for subsequent stimuli.

One embodiment incorporates a massage chair inside an egg shaped pod that uses its walls as a screen for VR projection while the pod delivers g forces that are associated with the projected images. VR may be fitted in a module separate from the electromagnetic coil pods. A device station, when desired, may include back-up health features such as humidity control, an iv drip, HR and/or BP monitoring, etc. Accordingly, devices of this invention may be incorporated into or associated with larger or accessory structures or features. For example VR or ER (Enhanced Reality) may feature components reminiscent of the subject's past experiences. For example, one or more sensations, cognitive or physical environments, stress conditions, etc., may be suggested or duplicated to mimic a calm or stress as determined by the practitioner to guide the brain towards achieving desired therapeutic effect.

The present invention provides improvements over previous therapies that have incorporated magnetism in attempts to correct or ameliorate undesirable alterations or abnormalities in brain functions. Early attempts to correct brain anomalies used static DC magnetic fields. The surface stimulations common to the art while directed at a particular portion of the head of brain were not focused through a magnetic return path. The incorporation of at least two interplexed magnetic stimulation pathways provides accurate targeting specific to the intended zone/area. Three interplexed circuits provides an extra margin of safety with regard to off-target stimulation. If desired, additional circuits can be included in the device. Sometimes not all the circuits will be active in a given session. In situation with dormant circuits, the active circuits would be those positioned with optimal pathways to the targeted zone(s) are selected for activation in a particular session. Rather than anticipate providing hardware that allows for positioning by selecting specific pairs, the device can be configured with movable coils that may be set prior to a session or might be tracked for movement during a session. Multiplexing becomes more difficult with each additional channel requiring shorter bursts from each channel. In some cases, one or two circuits may be favored with a third, fourth, fifth, etc., channel being activated fewer times than the preferred channel(s). For example, an activation order may be: 1, 2, 3, 2, 5, 1, 2, 7, 6, 2, 1, 2, 3 or any variation as determined by the AI algorithm using feedback for the particular subject session and device.

Coils may be refined by insertion of a ferro-active pin or plug projection in the center of the coil. In one example, a quadrupole is situated to provide a magnetic beam. The projection outcrops the magnetic field to a pinpoint dimension. The projections, rather than the coils contact or are made closely proximate to the scalp for a more reliable focused stronger signal.

### EXAMPLE

A subject presents with a behavioral abnormality or functional deficit relating to nervous function originating in the brain. A brief medical and/or experiential history is referenced or obtained. The subject's head is brought into an analysis zone either by moving the subject or by moving an imaging device or system to surround the head in a manner to provide 3-D functional imaging of brain activity. The history may be instructive in efficient placement of imaging tools. An image is obtained to indicate zones or areas of the brain where cellular activity is abnormal. An outside stimulus may be applied to potentially activate or amplify the historical disorder. For example, visual images may be used to create perceptions that might alter brain activity to emphasize or increase the abnormal activity. For example, an instance (visual, sound, olfactory, etc.) shown in the subject's history to predispose a panic attack may be presented to identify the areas activated and thus to be targeted for desensitization. Conversely, a calming, e.g., flowing image or calming sound may be used to strengthen the subject's capacity to remain sedentary for the therapy.

The imaging device can also incorporate the magnetic coils used to deliver therapy. A different imaging system may be used during the therapeutic sessions. Preferably, the session feedback imagers are equipped with filtering hardware or software to separate the magnetic therapy from the brain activity signals. Feedback may be coincident with the therapeutic signals and continue during breaks such as interpulse intervals. When feedback is desired during the pulse phase, not the interpulse phase, the frequencies of the therapeutic pulsing should be outside those of brainwaves. For example, some epilepsies feature a 3 Hz neuronal activity as monitored on EEG. When feedback is to be collected during pulsing sessions, all the relevant frequencies of the active device must be selected to avoid interference.

The orthogonally situated coils that may feature accessory hardware to focus or concentrate a magnetic beam are not universally activated. The system comprises at lease two sets of coils so that the signals can be made to cross at the targeted zone. Three pairs of coils can be used to more robustly concentrate therapeutic stimulus on the zone coordinates determined by the algorithm developed by the embedded AI from the subject histories, imaging and coil characteristics.

A preferred AI continuously adapts with each subject session. Early applications with devices of the invention use Ai based on data available, including contemporary and historical literature to identify targeted areas and stimulus formats. Data from subject sessions are periodically added into the learning set(s). Sets may comprise subsets with weightings of data results specific to, e.g., to age, diagnosis, handedness, etc. The AI operates in accord with conventional machine learning paradigms like LLM where patterns are learned to predict subsequent items. The electronic signals from the brain act as words in do on the LLM models. The models predict the next word (data block or signal) based on previous patterns. Simply described, the AI looks at its learning library, identifies features relevant to current signal inputs from the subject, e.g., EEG readings, medical history, historical data from that subject and similar subjects, diagnoses, etc., then queries the AI system with suggested algorithm steps and analyzes predicted outcomes. In preferred practice multiple initial steps are proposed to the AI system which deselects initial steps with less advantageous outcomes. The AI proceeds step-by-step to form the algorithm that activates the stimulus coils. Where coincident feedback is available, the AI continuously adapts or updates the algorithm output to optimize subject treatment outcome. The AI operates for each subject session and when a subject is slated for multiple therapeutic sessions is better enabled as experiences are incorporated into the AI protocol. The AI thereby incorporates past results to indicate expectations in sessions to come. Past and continuing results are considered as the current session therapeutic algorithmic protocol is delivered to the stimulus coils. In a preferred practice, feedback signals reporting neurologic response to the stimuli are compared with content in a learned database relevant to the instant subject, with individual subject historical progression, and with in session feedback. Such broad considerations by the AI for the instant algorithms optimizes each session for the subject. For example, the AI may produce algorithms during a session that might skip or repeat stimulating a zone or activating a coil pair in session when feedback determines the optimal stimulation pattern8ng in the time allotted for the session and planned future sessions.

Some embodiments may comprise at least two sets of coils so that the signals can be made to cross at the targeted zone without refitting the device on the scalp or moving coils to focus through different cranial angles. E.g., three pairs of coils might be used to more robustly concentrate therapeutic stimulus on the zone coordinates determined by the algorithm developed by the embedded AI from the subject histories, imaging and coil characteristics while feeding less flux in in undesired paths than would occur if a single flux pathway were repetitively activated.

Other formats for employing multiple paths to focus on a target are available. For example, a helmet or structure covering or surrounding the skill can be outfitted with a system to allow repositioning of coils or coil pairs around brain. The movement may be controlled as desired by the practitioner, e.g., gear-like tracks inside a cap or frame, cables to move coils along tracks, magnetic or electromagnetic pulses to arithmetically drive a coil to its new desired location over a targeted zone on the skull.

Coils can also be outfitted on robotic arm-like structures. The controlling algorithm lifts the coil(s) off the head, transfers the coil(s) to a zone above the new desired path site, and lowers the coil(s) to the skull prior to activating the coils.

Additional embodiments may feature orthogonally situated coils that may feature accessory hardware to focus or concentrate a magnetic beam to ensure other unintended coils are not unintentionally activated. The system comprises at lease two sets of coils so that the signals can be made to cross at the targeted zone. Three pairs of coils can be used to more robustly concentrate therapeutic stimulus on the zone coordinates determined by the algorithm developed by the embedded AI from the subject histories, imaging and coil characteristics.

The algorithm adjusts the timing—length of pulsing for each pair, the switching between pairs, the intensities of each pair, frequencies of pulses within a cluster of pulses, recovery intervals, the pulse amplitudes and shape e.g., just positive at a given coil and negative at the other—sinusoidal, ramped, squared, etc., frequency of pulsing, number of pulses for each pulse session for each pair, activation—length of time each packet of pulses is delivered from each coil, the amplitude of each stimulus signal within a pulsing cluster, the switch rate between coil pairs, etc. Each pair may be separately and dynamically controlled, For example pair x may deliver pulses at an amplitude a for ^{˜}20 msec, before switching to pair y that may deliver pulses at an amplitude b for 3 msec, followed by switching to pair z that may deliver pulses at an amplitude c for ^{˜}12 msec, before switching to pair y at amplitude b′ for 11 msec. The stimulating paradigm comprises rapid switching between orthogonally arranged magnetic circuits, where each pair undergoes of magnetic circuits undergoes on-off cycling in concert with other orthogonally arranged pair(s) at a rate where the targeted area receives the signals from a second pair while the tissue is responding to the signal from the first pair. The switching of the physical signal can be more rapid than the biologic signal response. A second cycling and subsequent cyclings amongst the pairs often immediately follows. Rapid switching between coil pairs allows continuous delivery of stimulus to the target while delivering below threshold energies to the non-targeted zones that may be in the path of a single pair. Pulsing time or cluster time length for each pair activated is set by the AI in adjusting the algorithm. For example, a first pulse duration may be 400 msec, a second 300 msec, a third 122 msec. An interpulse or cluster recovery interval may be called for after a preprogrammed period of time or a max duration determined in the algorithm based on previous experiences, patient comfort or safety concerns. For example, an interpulse/intercluster interval may be mandated to occur after 3 sec, 2 sec, 1 sec, 800 msec, 736 msec, 660 msec, 500 msec, 400 msec, 370 msec, 333 msec, 300 msec, 250 msec, 220 msec, 200 msec, 100 msec, 50 msec, 30 msec, 20 msec, 18 msec, 15 msec, 12 msec, 10 msec, 8 msec, 5 msec, 3 msec, etc. These expressed numbers are arbitrarily selected as examples; the algorithm will instruct he device with specific timelengths, frequencies, amplitudes, etc. The arbitrariness of the specific instructions, of course, is not restricted to integer numbers. The algorithm instructions may include or be overridden by safety criteria, e.g., length of a pulsing session, temperature measured on the subject, noise or movement made by the subject, amplitudes of a signal, temperature in the circuitry, etc. Control circuitry is available that can deliver pulses with μsec up to several second durations. Pulse frequencies of any fraction of the duration are therefore available in a range from the μsec to several seconds. Frequencies in a midrange of these extremes more closely match observed biological frequencies at the cellular level. The Litz conductors minimize heating for longer stimulus sessions. Interpulse intervals therefore can be selected by the AI to optimize therapeutic effect for the subject without significant restrictions relating to heat discomfort or to damage to tissue or equipment of concern when using conventional TCS and/or rTMS circuitry.

The therapeutic methods of the present invention feature a diagnostic pre-screen. The pre-screen may be multi-dimensional, I.e, involving one or more targeting diagnostics such as MRI, PET, 3D-EEG, or other imaging protocols as well as the pre-screen for potentially problematic material. The pre-screen may involve a rest state and/or may involve stimuli to assist diagnostics.⁹ 9. The present invention employs a variation on these methods but must be cognizant of potential unknown, implanted, or unrecovered metal substances that might be moved or drawn by the magnetic forces. Before application of the therapeutic fields a scan is advised to identify foreign or magnetically influential materials that may affect or be affected by the therapeutic applications of stimulating magnetic currents.

The AI is configured to adjust the algorithms as suggested by the feedback data. For example, an intensity/amplitude may be adjusted to improve balance of signals at the target; the pulse frequency can be adjusted to better activate or suppress cellular activity at the target and may be configured to target specific cell types at the target; the number of pulses from each node and in some cases pulse frequency or shape may be varied during a session. The duration of a pulse session as controlled by the algorithm may involve activating a pair a single time, two times, 3, 4, 5 6, 7, 8, etc., times in a session. Pulse sessions are separated in time by interpulse intervals, also adjusted in real-time by the AI as it continuously updates the algorithm based on the feedback data. The sessions are interrupted by interpulse intervals that allow the tissues to reset, e.g., strengthen or reestablish ion gradients, turnover receptors, recycle neurotransmitters, etc. Interpulse. A distinct series of coil pairs may be activated at different cycles and/or selected in session to more efficiently align with axonal direction.

A preferred device configuration features multiple pairs, e.g., 3 pairs of orthogonal coils with Litz wiring to reduce heating from proximity effect and skin effect that increase with higher frequencies. An alternative configuration can use hollow, e.g., square, conductive, e.g., silver, copper, etc., windings to reduce proximity/skin effects to reduce need for cooling. Cooling with purified, deionized water, e.g., RO18 water with anti-leach additive to protect conductor and avoid ions escaping into the low conductance—high resistance water. An amorphous metal band/ribbon is the preferred conductor for connecting the coils of each pair. Several cobalt based amorphous metallic alloys are available for this job and can be formed form flattened conductive low reluctance connectors and coils. Ribbons may be stacked for higher effect. The current transmits between the coils to produce currents in the opposing coils and induce intracranial currents. The orthogonally arranged coil pairs are used to target the magnetic fluxes and induced currents to specific zones in the brain. A flux concentrator is useful for concentrating and focusing the magnetic forces. High frequency switching and relatively high powers for deeper penetrations is meticulously controlled using algorithms that incorporate frequency, distance and transmission times such that the signals are constructive rather than destructive at the target. Lengthy pulsation streams are enabled by the low resistance in the energizing connections in the circuits. Preferably, cabling uses multiple thin wire strands individually insulated and twisted or woven together in a Litz Wire format to distribute the electric current equally across the wire strands. This minimizes skin effect resistance and reduces heat generation in the cabling.

Pulsation clusters of msec up to several seconds in length, e.g., 20 msec to 20 sec are alternated with rest periods where the cells' interfaces recover. These limits can be surpassed under direction of the AI directed algorithm, but preferably extremes in these and other parameters under control of the operating algorithm are flagged for operator attention and opportunity to override. Membrane receptors are internalized and/or recycled, Transmitter chemicals are cleared. The releasing termini are resupplied with vesicles for the next round. The rest periods are adjusted to concentrate effects on the desired cell type, e.g., astrocytes, ependymal cells, and microglial cells. Microglial cells are rapidly fatigued, that is once activated return to the original state is more lengthy than that of neurons. Astrocytes tend to show a recovery time in between. Short rests concentrate on neuronal; activity. Microglial recovery can stretch to hours; and astrocyte excitement may be optimized by interpulse intervals of several minutes. For each individual and each therapy for each individual the pulse frequency, interpulsing durations and intersession timing should be confirmed with one or more of the imaging techniques. General acceptance of the subject to length of treatment sessions, time between treatments and ability to attend the treatments should also be considered in therapy.

1. A transcranial stimulation device comprising:
a magnetic flux generation circuit comprising at least two (one pair of) conductive coils;
a data processor interface for supplying output to input to said flux generation circuit, an imaging tool that reports location of activity within the brain
said input comprising instruction to control activation of said flux generation circuit;
said data processor comprising capacity to harbor at least one AI routine that incorporates location information of said flux generation circuit, report from said imaging tool, experiences from at least one subject reacting to stimulation from said magnetic flux generation circuit;
said AI routine instructing at least a portion of said transcranial stimulation device to generate at least one algorithm to process said output;
an input to said data processor, said input configured for receipt of data associated with brain activity.

2. The transcranial stimulation device of claim 1 wherein said data associated with brain activity comprises data outputted by said imaging tool.

3. The transcranial stimulation device of claim 1 wherein said imaging tool comprises three dimensional EEG.

4. The transcranial stimulation device of claim 1 wherein said at least one pair of conductive coils are connected with Litz wire.

5. The transcranial stimulation device of claim 1 comprising at least a second pair of conductive coils.

6. The transcranial stimulation device of claim 5, wherein at least one member of at least one said pair of conductive coils is movable or retractable.

7. The transcranial stimulation device of claim 1 configured in a support in a head covering or cap format.

8. The transcranial stimulation device of claim 7, further comprising a track or arm that is interfaced with said at least one pair of conductive coils to move or retract said at least one pair of conductive coils.

9. The transcranial stimulation device of claim 1 further comprising a virtual reality (VR) stimulatory component, said VR component eliciting neuro activity in at least one zone of stimulatory interest.

10. A Repetitive Transcranial Magnetic Stimulation (rTMS) device comprising:
a plurality of orthogonally arranged magnetic circuits, said circuits each comprising a pair of electromagnetic coils;
a sensing apparatus to detect electrical activity,
wherein said sensing apparatus is provided with screening hardware or software to filter out or ignore outputs from said orthogonally arranged magnetic circuits to provide data of signals emanating from adjacent tissue electrical activity,
wherein said data emanating from adjacent tissue electrical activity comprises indications of strength, frequency and source of said signals;
a processor that receives said data for incorporation into a machine learning process to develop an Artificial Intelligence (AI) algorithm output,
said AI algorithm output capable of adjusting the activation of each of said plurality of orthogonally arranged magnetic circuits to focus signal from each of said activated magnetic circuits to concentrate on a targeted zone within a void between opposing poles of said magnetic circuits.

11. The device of claim 10 comprising at least three orthogonally arranged magnetic circuits.

12. The device of claim 10 wherein at least a first of said plurality of orthogonally arranged magnetic circuits is positionally adjustable with respect to a second of said plurality of orthogonally arranged magnetic circuits.

13. The device of claim 10 wherein electrical circuitry of said plurality of orthogonally arranged magnetic circuits comprises Litz wire.

14. The device of claim 10 comprising a ferro active projection from a pole of at least one said orthogonally arranged magnetic circuit, said projection concentrating magnetism effect from said pole.

15. The device of claim 10 further comprising a flux concentrator associated with at least one pole of said orthogonally arranged magnetic circuits.

16. The device of claim 10 further comprising a hood encompassing said plurality of orthogonally arranged magnetic circuits.

17. The device of claim 16 further wherein a first pair of said plurality of orthogonally arranged magnetic circuits is adjustable in space with respect to a second pair of said plurality of orthogonally arranged magnetic circuits.

18. The device of claim 10 further comprising a second sensor, said second sensor capable of measuring magnetic flux produced by at least one of said orthogonally arranged magnetic circuits, said second sensor interfaced with said AI algorithm capable of adjusting the activation to control delivery of said magnetic flux in accordance with algorithmic direction.

19. The device of claim 10 further comprising a sound generates that feeds ultrasonic energy into said targeted zone.

20. A hyperbaric chamber comprising a device of claim 10.

21. A method for inducing modification in neurocircuitry, said method comprising:
analyzing a subject brain to identify a zone or area of abnormal function;
introducing coordinates of said zone or area into said processor of claim 9;
instructing said processor to create or access an AI algorithm specific to said subject brain;
stimulating said subject brain in accord with said algorithm;
collecting response data from said zone or area;
adjusting said algorithm based on said response data; and
stimulating said subject brain in accord with said adjusted algorithm.

22. The method of claim 21 wherein said analyzing comprises imaging brain function using a method selected from the group consisting of: MRI, PET, 3D-EEG, CT, PET-CT, and Doppler.

23. The method of claim 21 wherein said stimulating comprises rapid switching between at least two of said plurality of said orthogonally arranged magnetic circuits, said rapid switching comprising on-off cycling of said magnetic circuits at a rate <20 msec.

24. The method of claim 21 wherein said algorithm activated less than a totality of said plurality of orthogonally arranged magnetic circuits.

25. The method of claim 21 wherein said stimulating comprises rapid switching of said stimulating between said plurality of orthogonally arranged magnetic circuits on a time scale resulting in a zone at the intersection of the intracranial stimulating from at least two of said plurality of orthogonally arranged magnetic circuits receiving a signal post said rapid switching before a response at said zone to a signal prior to said rapid switching terminates.
